(19) United States
(12) Patent Application Publication
(10) Pub. No.: US 2014/0309308 A1
(43) Pub. Date: Oct. 16, 2014

(54) DEPILATORY COMPOSITIONS

(71) Applicant: RECKITT & COLMAN (OVERSEAS) LIMITED, Berkshire (GB)

(72) Inventor: Jane Evison, Hull (GB)

(73) Assignee: Reckitt & Colman (Overseas) Limited, Berkshire (GB)

(21) Appl. No.: 14/357,782

(22) PCT Filed: Nov. 13, 2012
(86) PCT No.: PCT/GB2012/052814

§ 371 Date: May 13, 2014

(30) Foreign Application Priority Data
Nov. 14, 2011 (GB) 1119601.1

(51) Int. Cl. A61K 8/44; A61K 8/46; A61K 8/42; A61Q 9/04

(52) U.S. Cl. CPC A61K 8/44; A61Q 9/04; A61K 8/46; A61K 8/42
USPC 514/565

(57) ABSTRACT

The present invention is directed to a depilatory composition which comprisesa depilatory compound having a thiol group; and at least 0.1 wt % of arginine based on the total weight of the composition.

The present invention relates to a depilatory composition comprising a depilatory compound having a thiol group, a cross-linked polymer comprising maleic anhydride and arginine, and to a method of depilation using such a composition. In particular, the present invention relates to depilatory gel which has significantly reduced irritation.

Compositions for removing superfluous body hair which include a compound that degrades hair keratin are well known. After the composition has been applied it is allowed to remain on the skin to degrade the hairs and is then removed together with the degraded hairs.

Depilatory compositions of this type typically comprise depilatory compounds which have a thiol group such as potassium thioglycolate, which typically contain sodium hydroxide to provide a high pH. However, there is a significant disadvantage in using these compounds in that they can irritate and even damage the skin as a result of the high pH (usually above 12).

WO 9844898 describes depilatory compositions with reduced malodour where very low levels of arginine are used in conjunction with polyethyleneimine to effect buffering of the composition.

Surprisingly, it has been found that addition of arginine over and above the levels used for buffering greatly reduces the skin irritation.

According to a first aspect of the present invention, there is described a depilatory composition which comprises:
a depilatory compound having a thiol group; and,
at least 0.1 wt % of arginine based on the total weight of the composition.

Preferably the depilatory composition as hereinbefore described comprises between 0.5 and 5 wt % arginine based on the total weight of the composition.

More preferably the depilatory composition as hereinbefore described comprises between 2.0 and 4.0 wt % arginine based on the total weight of the composition.

In one aspect of the invention, the depilatory product can be in the form of a gel.

When the depilatory product according to the present invention is in the form of a gel, it preferably comprises a cross-linked copolymer comprising maleic anhydride.

Preferably the cross-linked polymer is present in an amount of from 1.5 to 5 wt % based on the total weight of the composition; more preferably in an amount of from 2 to 3 wt % based on the total weight of the composition.

In a preferred depilatory composition as hereinbefore described the cross-linked copolymer further comprises methylvinylether.

More preferably, the cross-linked polymer comprised by the depilatory composition as hereinbefore described is poly(methylvinyl ethermaleic anhydride) co-polymer crosslinked with 1,9 decadiene.

In a further aspect of the invention, the depilatory product can be in the form of a cream.

The depilatory compound is preferably present in the composition in an amount of from 2 to 10 wt % based on the total weight of the composition, more preferably from 3 to 7 wt %.

The depilatory composition further comprises a salt selected from di- and tri-valent salts, such as magnesium chloride, calcium chloride ammonium chloride, magnesium sulphate, calcium sulphate, aluminium sulphate, magnesium carbonate, calcium sulphate, calcium carbonate. A preferred salt is magnesium chloride. In an alternative embodiment the composition can comprise a salt extract, such as dead sea salts.

The depilatory compound having a thiol group may be any compound capable of degrading keratin. Examples of such compounds are potassium thioglycolate, dithioerythritol, thioglycerol, thioglycol, thioxanthine, thiosalicylic acid, N-acetyl-L-cysteine, lipoic acid, sodium dihydrolipoate 6,8-dithioocatanoate, sodium 6,8-diothioocatanoate, a hydrogen sulphide salt, thioglycolic acid, 2-mercaptopropionic acid, 3-mercaptopropionic acid, thiomalic acid, ammonium thioglycolate, glyceryl monothioglycolate, monoethanolamine thioglycolate, monoethanolamine thioglycolic acid, diammoniumdithiodiglycolate, ammonium thiolactate, monoethanolamine thiolactate, thioglycolamide, homocysteine, cysteine, glutathione, dithiothreitol, dihydrolipoic acid, 1,3-dithiopropanol, thioglycolamide, glycerylmonothioglycolate, thioglycolhydrazine, keratinase, guanidine thioglycolate, calcium thioglycolate andor cysteamine. A single compound or a mixture of two or more compounds may be used.

Preferably the depilatory compound is potassium thioglycolate.

The depilatory composition may, if desired, comprise further components.

The depilatory composition may, for example, comprise components which accelerate the keratin degradation reaction such as urea, thiourea, dithioerythritol, dimethyl isosorbide (DMI), ethoxydiglycol (Transcutol) or methyl propyl diol (MT diol). The composition desirably comprises up to 15 wt % of the accelerator based on the total weight of the composition, preferably from 0.2 to 15 wt % and more preferably from 0.5 to 10 wt %.

The composition may comprise a source of alkalinity. This may include hydroxides, such as hydroxides of alkali and alkaline earth metals. Suitable hydroxides include sodium hydroxide, potassium hydroxide, calcium hydroxide and magnesium hydroxide. Preferably, potassium hydroxide is employed, optionally together with calcium hydroxide. The source of alkalinity (e.g. calcium hydroxide) may be present in an amount of 0.1 to 10 weight %, preferably 1 to 6 weight %, for example 2 to 5 weight % of the depilatory cream composition.

The depilatory composition preferably has a pH of greater than 7, for example, 9 to 12.7.

Preferably the alkali metal hydroxide is present in an amount of at least 0.001 mol/100 g of composition, preferably in an amount of at least 0.01 mol/100 g of the composition.

The depilatory composition of the present invention may comprise water, suitably in an amount of at least 40 or 50 wt % based on the total weight of the composition, more preferably at least 60 wt %.

The depilatory composition may further include a humectant. Suitable humectants include polyols, such as glycerine, propylene glycol and butylene glycol. Glycerine is preferred. The humectant may be present in an amount up to 10 wt %, preferably 0.5 to 5 wt %.

The depilatory composition may further include an emollient such as an oil or a wax. A preferred oil emollient is mineral oil. A preferred wax emollient is ceteareth-20. The depilatory cream composition may include a mixture of an oil and a wax as a combined emollient.

Other suitable components which may be incorporated into the composition include a buffer such as sodium silicate or magnesium silicate, fragrances such as lotus milk, and a chelating agent such as sodium gluconate.

In a particularly preferred aspect of the present invention, there is provided a depilatory composition in the form of a gel comprising:

(a) 1-5% w/w poly(methylvinylethermaleic anhydride) co-polymer crosslinked with 1,9 decadiene;

(b) 0.5-5% wt arginine;

(c) 0.01-1% w/w an inorganic salt;

(d) 0-10% w/w glycerine;

(e) 5-10% w/w urea;

(f) 0.01-1% w/w sodium gluconate;

(g) 0.01-1% w/w cosmetic extract;

(h) 2-15% w/w aqueous potassium thioglycolate solution;

(i) 0-5% w/w sodium silicate solution;

(j) 1-5% w/w potassium hydroxide; and

(k) water up to 100%.

Especially, the depilatory composition comprises:

(a) 2.2% w/w poly(methylvinyl ethermaleic anhydride) co-polymer crosslinked with 1,9 decadiene;

(b) 3.0% wt arginine;

(c) 0.1% w/w magnesium chloride;

(d) 3% w/w glycerine;

(e) 1% w/w urea;

(f) 0.1% w/w sodium gluconate;

(g) 0.1% w/w cosmetic extract;

(h) 5% w/w potassium thioglycolate;

(i) 2.5% w/w sodium silicate solution;

(j) 8% w/w potassium hydroxide 50% solution; and

(k) water up to 100%.

Particularly preferred is a depilatory composition which consists essentially of components (a)-(k).

Example embodiments of the present invention will now be described.

### COMPARATIVE EXAMPLE 1

| Ingredient (Trade Name) | % w/w |
|---|---|
| Stabileze QM | 2.2 |
| Magnesium Chloride | 0.05 |
| Glycerine | 5 |
| Urea | 8 |
| Sodium Gluconate | 0.1 |
| Cosmetic extract | 0.1 |
| Potassium thioglycolate solution (43 wt %) | 12 |
| Sodium Silicate solution | 2.5 |
| KOH 50% sol. | 7.7 |
| Deionised Water | to |
| | 100% |

### Invention Example 1

| Ingredient (Trade Name) | % w/w |
|---|---|
| Stabileze QM | 2.2 |
| Glycerine | 3 |
| Urea | 0.5 |
| Sodium Gluconate | 0.1 |
| Lotus Milk extract | 0.1 |
| Potassium thioglycolate solution (43%) | 12 |
| Sodium Silicate solution | 2.5 |
| KOH 50% sol. | 8.2 |
| Deionised Water | to 100% |
| L-Arginine | 3 |
| Fragrance | 0.5 |
| Cosmetic extract | 0.1 |
| Colorant | q.s. |

### Invention Example 2

| | % w/w in |
|---|---|
| Ingredient (Trade Name) | Formula |
| Deionised water | to 100% |
| Potassium Thioglycolate soln 43% | 12 |
| Potassium Hydroxide 50% | 8.5 |
| Glycerin | 3 |
| Sodium Silicate | 2.5 |
| Stabilize QM | 2.2 |
| Urea | 0.5 |
| fragrance | 0.5 |
| Cosmetic extract | 0.1 |
| Sodium Gluconate | 0.1 |
| Magnesium Chloride | 0.05 |
| Colorant | q.s. |
| Thick Mineral Oil | 2 |
| L-Arginine | 3 |

### Invention Example 3

| | % w/w in |
|---|---|
| Ingredient (Trade Name) | Formula |
| Deionised water | to 100% |
| Potassium Thioglycolate 43% | 12 |
| Potassium Hydroxide 50% | 8.5 |
| Glycerin | 3 |
| Sodium Silicate | 2.5 |
| Stabilize QM | 2.2 |
| Urea | 0.5 |
| fragrance | 0.5 |
| Cosmetic extract | 0.1 |
| Sodium Gluconate | 0.1 |
| Magnesium Chloride | 0.05 |
| Colorant | q.s. |
| Thick Mineral Oil | 0.6 |
| Cetearyl Alcohol | 1 |
| Ceteareth-20 | 0.4 |
| L-Arginine | 3 |

### Invention Example 4 (Cream Product)

| | % w/w in |
|---|---|
| Ingredient (Trade name) | Formula |
| Thick mineral oil | 4.8 |
| Sorbitol | 1 |
| Glycerin | 1 |
| Cetearyl Alcohol | 4.4 |
| Ceteareth 20 | 1.76 |
| Calcium Hydroxide | 3.56 |
| Talc | 2 |
| Magnesium Trisilicate | 0.5 |
| Sodium Gluconate | 0.1 |
| cosmetic extract | 0.1 |
| Colour paste | 0.6 |
| Laponite XLG | 0.2 |
| Urea | 8 |
| Acrylate copolymer | 0.1 |
| Fragrance | 0.4 |
| silica gel | 0.025 |
| Potassium Thioglycolate 43% | 12.9 |
| Potassium Hydroxide 50% | 0.5 |
| L-Arginine | 1 |
| DI water | to 100% |

Stabileze QM is a cross-linked methylvinylethermaleic anhydride co-polymer.

Compositions according to the examples were compared with gels not comprising arginine to demonstrate the ability of the compositions of the present invention to reduce irritation when in contact with human skin. The compositions tested are shown in Table 1 below, and the results are shown in the Table 2.

An in vitro method was used, wherein assays of cells were treated with different example products.

The assay measured in each case the time taken for the cells to reduce by 50% (ET-50). This is the key measure for anti-irritancy as it is a direct measure of the survival of cells, as cells are destroyed by the high pH of the composition.

The results given here are based on several measurements at different exposure times.

TABLE 1
Examples for in vitro testing (along with Comparative
Example 1, number (4) in Table 2)

| | Dry | L-Arginine | |
|---|---|---|---|
| | standard | Sensitive | Sensitive |
| | Base (1) | Gel base (2) | Gel Base (3) |
| Stabileze QM | 2.2 | 2.20 | 2.20 |
| L-Arginine | 0 | 3 | 0 |
| Magnesium Chloride | 0.05 | 0.10 | 0.25 |
| Glycerine | 3.0 | 3.00 | 3.00 |
| Urea | 1.0 | 1.00 | 1.00 |
| Sodium Gluconate | 0.1 | 0.10 | 0.10 |
| TGK 31% | 12.0 | 10.00 | 10.00 |
| Sodium Silicate | 2.5 | 2.50 | 2.50 |
| KOH 50% sol | 8.0 | 8.00 | 8.00 |
| DI H20 | to 100% | to 100% | to 100% |
| Fragrance | 0.5 | 0.50 | 0.50 |
| Cosmetic extract | 0.1 | 0 | 0 |
| Symrelief | 0.05 | 0 | 0 |
| Soothex CG | 1.0 | 0 | 0 |
| Colorant | q.s | q.s. | q.s. |
| Cosmetic Extract | 0 | 0.10 | 0.10 |

1. A depilatory composition comprising:
a depilatory compound having a thiol group; and,
at least 0.1 wt % of arginine based on the total weight of the composition.

2. The depilatory composition according to claim 1 further comprising a cross-linked copolymer comprising maleic anhydride.

3. The depilatory composition according to claim 1 comprising between 0.5 and 5 wt % arginine based on the total weight of the composition.

4. The depilatory composition according to claim 1 comprising between 2.0 and 4.0 wt % arginine based on the total weight of the composition.

5. The depilatory composition as claimed in claim 2, wherein the cross-linked polymer is present in the composition in an amount of from 1.5 to 5 wt % based on the total weight of the composition; and
wherein the composition is in the form of a gel.

6. The depilatory composition as claimed in claim 5, wherein the cross-linked polymer is present in the composition in an amount of from 2 to 3 wt % based on the total weight of the composition.

7. The depilatory composition as claimed in claim 2, wherein the cross-linked copolymer further comprises methylvinylether; and
wherein the composition is in the form of a gel.

8. The depilatory composition as claimed in claim 2, wherein the cross-linked polymer is poly(methylvinyl ethermaleic anhydride) copolymer crosslinked with 1,9 decadiene; and
wherein the composition is in the form of a gel.

9. The depilatory compound according to claim 1 which is in the form of a cream.

10. The depilatory composition as claimed in claim 1 further comprising a salt selected from di- and tri-valent salts.

11. The depilatory composition as claimed in claim 10 wherein the salt is magnesium chloride.

12. The depilatory composition as claimed in claim 1, wherein the thiol group comprises a compound capable of degrading keratin.

13. The depilatory composition as claimed in claim 10, wherein the depilatory compound is selected from the group consisting of potassium thioglycolate, dithioerythritol, thioglycerol, thioglycol, thioxanthine, thiosalicylic acid, N-acetyl-L-cysteine, lipoic acid, sodium dihydrolipoate 6,8-dithioocatanoate, sodium 6,8-diothioocatanoate, a hydrogen sulphide salt, thioglycolic acid, 2-mercaptopropionic acid, 3-mercaptopropionic acid, thiomalic acid, ammonium thioglycolate, glyceryl monothioglycolate, monoethanolamine thioglycolate, monoethanolamine thioglycolic acid, diammoniumdithiodiglycolate, ammonium thiolactate, monoethanolamine thiolactate, thioglycolamide, homocysteine, cysteine, glutathione, dithiothreitol, dihydrolipoic acid, 1,3-dithiopropanol, thioglycolamide, glycerylmonothioglycolate, thioglycol-hydrazine, keratinase, guanidine thioglycolate, calcium thioglycolate and cysteamine.

14. The depilatory composition as claimed in claim 13, wherein the depilatory compound is potassium thioglycolate.

15. The depilatory composition as claimed in claim 1, wherein the depilatory compound is present in the composition in an amount of from 2 to 10 wt % based on the total weight of the composition.

16. The depilatory composition as claimed in claim 1, wherein the depilatory compound is present in the composition in an amount of from 3 to 7 wt %.

17. The depilatory composition as claimed in claim 1 further comprising a component which accelerates the keratin degradation reaction.

18. The depilatory composition as claimed in claim 17, wherein the composition comprises up to 15 wt % of the accelerator based on the total weight of the composition.

19. The depilatory composition as claimed in claim 2 comprising:
1-5% w/w poly(methylvinyl ethermaleic anhydride) co-polymer crosslinked with 1,9 decadiene;
0.5-5% wt arginine;
0.01-1% w/w an inorganic salt;
0-10% w/w glycerine;
0-10% w/w urea;
0.01-1% w/w sodium gluconate;
0.01-1% w/w cosmetic extract;
2-10% w/w potassium thioglycolate;
0-5% w/w sodium silicate solution;
1-5% w/w potassium hydroxide; and
water.

20. The depilatory composition as claimed in claim 19, wherein the composition comprises:
2.2% w/w poly(methylvinyl ethermaleic anhydride) co-polymer crosslinked with 1,9 decadiene;
3.0% wt arginine;
0.1% w/w magnesium chloride;
3% w/w glycerine;
1% w/w urea;
0.1% w/w sodium gluconate;
0.1% w/w cosmetic extract;
5.2% w/w potassium thioglycolate;
2.5% w/w sodium silicate solution;
8% w/w potassium hydroxide 50% solution; and
water.

21. A depilatory consisting essentially of :
1-5% w/w poly(methylvinyl ethermaleic anhydride) co-polymer crosslinked with 1,9 decadiene;
0.5-5 wt arginine;
0.01-1% w/w an inorganic salt;
0-10% w/w glycerine;
0-10% w/w urea;
0.01-1% w/w sodium gluconate;
0.01-1% w/w cosmetic extract;
2-10% w/w potassium thioglycolate;
0-5% w/w sodium silicate solution;
1-5% w/w potassium hydroxide; and
water.

22. A depilatory consisting essentially of:
3.0% wt arginine;
0.1% w/w magnesium chloride;
3% w/w glycerine;
1% w/w urea;
0.1% w/w sodium gluconate;
0.1% w/w cosmetic extract;
5.2% w/w potassium thioglycolate;
2.5% w/w sodium silicate solution;
8% w/w potassium hydroxide 50% solution; and
water.

23. The depilatory composition as claimed in 10, wherein the salt is selected from the group consisting of magnesium chloride, calcium chloride, ammonium chloride, magnesium sulphate, calcium sulphate, aluminium sulphate, magnesium carbonate, calcium sulphate, and calcium carbonate.

24. The depilatory composition as claimed in claim 17, wherein the component which accelerates the keratin degradation reaction is selected from the group consisting of urea, thiourea, dithioerythritol, dimethyl isosorbide (DMI), ethoxydiglycol (Transcutol), and methyl propyl diol (MT diol).
